(19) United States
(12) Patent Application Publication
(10) Pub. No.: US 2023/0415330 A1
(43) Pub. Date: Dec. 28, 2023

(54) A BASE FOR SUPPORTING A SURGICAL ROBOT

(71) Applicant: CMR Surgical Limited, Cambridge (GB)

(72) Inventor: James Oliver Grant, Cambridge (GB)

(73) Assignee: CMR Surgical Limited, Cambridge, OT (GB)

(21) Appl. No.: 18/252,769

(22) PCT Filed: Nov. 12, 2021
(86) PCT No.: PCT/GB2021/052928

§ 371 Date: May 12, 2023

(30) Foreign Application Priority Data
Nov. 16, 2020 (GB) 2017988.3

(51) Int. Cl. B25J 9/00; A61B 34/30; B25J 5/00

(52) U.S. Cl. CPC B25J 9/0009; B25J 5/007; A61B 34/30

(57) ABSTRACT

A base for supporting a surgical robot includes a first planar member on which the surgical robot is mounted. A second planar member is pivotably coupled to the first planar member about a first axis. The first planar member and the second planar member are independently rotatable about the first axis so as to stabilise the base on an uneven floor surface.

### FIELD OF THE INVENTION

This invention relates to a base for a surgical robot that is configured to stabilise the surgical robot on an uneven surface.

### BACKGROUND OF THE INVENTION

Surgical robotic systems are increasingly becoming a more favourable option for performing operations on human patients, due to the shorter hospitalisation, faster recovery times and reduced scarring advantages that they present. A typical surgical robotic system comprises a surgeon's console, one or more robot arms, and one or more surgical instruments comprising 15 an end effector for attachment to the robot arms. Examples of suitable end effectors include surgical instruments and cameras. The one or more robot arms are operated using controllers located on the surgeon's console and are used to manipulate the position and orientation of their respective end effectors. Thus, there is a master-slave control relationship between the surgeon's console and the end effectors.

In a known surgical robotic system, each robot arm within the system is mounted on its own surgical cart. The robot arm and its corresponding cart can jointly be referred to as a surgical robot. Whilst the surgeon's console is a stationary member of the robotic system, the surgical robots are moveable by virtue of the surgical carts to which the robot arms mounted. That is, 25 the surgical carts allow for the robot arms to be moved around an operating theatre so that they can be positioned near a patient during preparations for surgery, and also allow for movement of the robots between operating theatres and hospital buildings. Once they have been moved to the desired position for a surgical procedure, the surgical robots are required to be secured in position for the duration of that procedure.

Once a surgical robot has been secured in its desired position, it is important to ensure the stability of this apparatus. A stable surgical robot should not tilt, turn or perform accidental movements during a surgical procedure. The stability of the surgical cart in particular is of utmost importance to the performance of the surgical robotic system because, when an end effector is in direct contact with the patient during a surgical procedure, any inadvertent movement of its respective surgical cart will be transmitted to the end effector via the robot arm. The likelihood of an inadvertent movement of the cart is increased when the surgical robot is positioned on an irregular or uneven surface. Thus, if the surgical cart is unable to maintain stability on an uneven surface then this could result in undesired movements of the end effector, which in turn could have catastrophic implications for the patient.

There is a need to provide an arrangement for optimising the stability of a surgical robot on an irregular or uneven surface.

### SUMMARY OF THE INVENTION

According to a first aspect, there is provided a base for supporting a surgical robot, the base comprising: a first planar member on which the surgical robot is mounted; and a second planar member pivotably coupled to the first planar member about a first axis; wherein the first planar member and the second planar member are independently rotatable about the first axis so as to stabilise the base on an uneven surface.

The first axis may lie on a plane that contains the first planar member.

The surgical robot may be mounted on an upper surface of the first planar member.

The first axis may lie on the upper surface of the first planar member.

The movement of the first planar member and the second planar member may be constrained such that they are displaceable relative to each other about only the first axis.

The first axis may be parallel to at least one distal edge of the first planar member.

The second planar member may comprise an upper surface that is aligned with the upper surface of the first planar member when the base is located on an even surface such that the upper surfaces of the first and second planar members form an upper surface of the base.

The upper surface of the base may be in the shape of a quadrilateral.

The at least one distal edge of the first planar member may be aligned with at least one distal edge of the second planar member.

The first planar member may be pivotably coupled to the second planar member by a bearing.

The base may comprise one or more apertures, such that a moveable element is able to extend through each aperture, each moveable element being configured to move the surgical robot.

The one or more apertures may comprise four apertures.

The first planar member may comprise a first limiting feature configured to interact with a corresponding second limiting feature located on the second planar member, the first and second limiting features being configured to interact to limit the rotation of the first and second planar members about the first axis.

The first planar member may comprise a pair of first limiting features and the second planar member may comprise a corresponding pair of second limiting features.

The first limiting feature may be a tab extending from the first planar member, and the second limiting feature may be an indent in the second planar member that is complementary to the tab.

The base may be coupled to a mechanical brake system.

The mechanical brake system may comprise a linear lifting column.

The base may further comprise a third planar member pivotally coupled to the first member about a second axis, wherein the first planar member and the third planar member are independently rotatable about the second axis.

The second axis may be perpendicular to the first axis.

According to a second aspect, there is provided a method for stabilising a surgical robot on an uneven surface, the method comprising: stopping the motion of the surgical robot relative to the surface; lowering a linear lifting column towards the surface, the linear lifting column being coupled to a base, the base comprising a first planar member on which the surgical robot is mounted and a second planar member pivotably coupled to the first planar member about a first axis, so that the base of the surgical robot contacts the surface; and when the base contacts the surface, performing a relative rotation of the first and second planar members about the first axis so as to stabilise the base on the uneven surface.

### BRIEF DESCRIPTION ON THE FIGURES

The present invention will now be described by way of example with reference to the accompanying drawings. In the drawings:

FIG. 1 illustrates the arrangement of a surgical robot;

FIGS. 2A and 2B illustrate an arrangement for securing a surgical robot in a desired position;

FIG. 3 illustrates a first example of a base for supporting a surgical robot;

FIG. 4 illustrates a second example of a base for supporting a surgical robot;

FIG. 5 illustrates the base of FIG. 4 in contact an uneven surface;

FIG. 6 illustrates a third example of a base for supporting a surgical robot;

FIG. 7 illustrates a method for stabilising a surgical robot on an uneven surface.

### DETAILED DESCRIPTION

The arrangement of a surgical robot to be implemented within a surgical robotic system is illustrated in FIG. 1. The surgical robot 100 comprises a robot arm 102 with a plurality of rigid limbs which are coupled together by a plurality of joints. The joints are configured to apply motion to the limbs. The robot arm 102 is coupled at its proximal end to a surgical cart 104 and at its distal end to a surgical instrument 106. The surgical instrument 106 is attached to the robot arm 102 by an attachment located at the distal end of the robot arm. The attachment comprises a drive assembly for driving the articulation of the surgical instrument 106.

The surgical instrument 106 comprises an end effector that is suitable for performing a surgical procedure. The end effector may take any suitable form. For example, the end effector may be smooth jaws, serrated jaws, a gripper, a pair of shears, a pair of scissors, a needle for suturing, a camera, a laser, a knife, a stapler, a cauteriser or a suctioner. The end effector may alternatively be an electrosurgical instrument such as a pair of monopolar scissors. The robot arm 102 transfers drive to the end effector via the drive assembly interface located within the attachment at the distal end of the arm. The robot arm 102 is actuated by a number of drive sources and sensors that are distributed within the arm. The drive sources can be controlled by software that is implemented in dependence on inputs from the sensors located within the arm, and from an operator that issues commands at a surgeon command interface. The surgeon command interface may form part of a surgeon's console.

The robot arm 102 is mounted on a surgical cart 104. In FIG. 1 the surgical cart 104 has one robot arm 102 mounted to it. In other examples, there may be more than one robot arm mounted to the surgical cart 104. The surgical cart 104 allows the surgical robot 100 to be moved with respect to the surface on which it is in contact. For example, the surgical cart 104 allows the surgical robot 100 to be moved along the floor of an operating theatre, and between operating theatres. The surgical cart 104 may also house electronic components that form the drive system of the robot arm 102 and its associated surgical instrument. As well as enabling movement of the surgical robot 100, the surgical cart 104 is also configured to secure the robot in a position that is desirable for a surgical procedure.

In addition to the robot arm 102, the surgical cart 104 is further coupled to a base 108 and one or more moveable elements 110. The moveable elements 110 are mechanical components that enable the movement of the surgical cart 104 relative to a floor surface. In one example, the moveable elements 110 are wheels. However, it will be appreciated that the moveable elements 110 may alternatively be any component that is capable of moving the surgical cart 104. The base 108 is connected to the lowermost surface of the surgical cart 104. The base 108 is configured as a flat plate and is therefore commonly referred to as a base plate. The base 108 may further comprise one or more apertures through which the moveable elements 110 can extend. Thus, the moveable elements 110 are not directly connected to the base 108 and can be moved independently of the base. Correspondingly, the base 108 can be moved independently of the moveable elements 110.

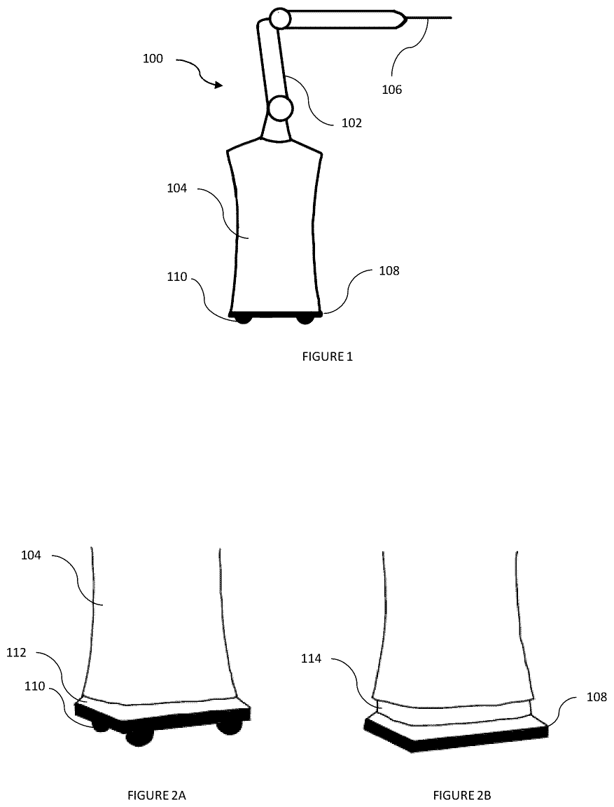

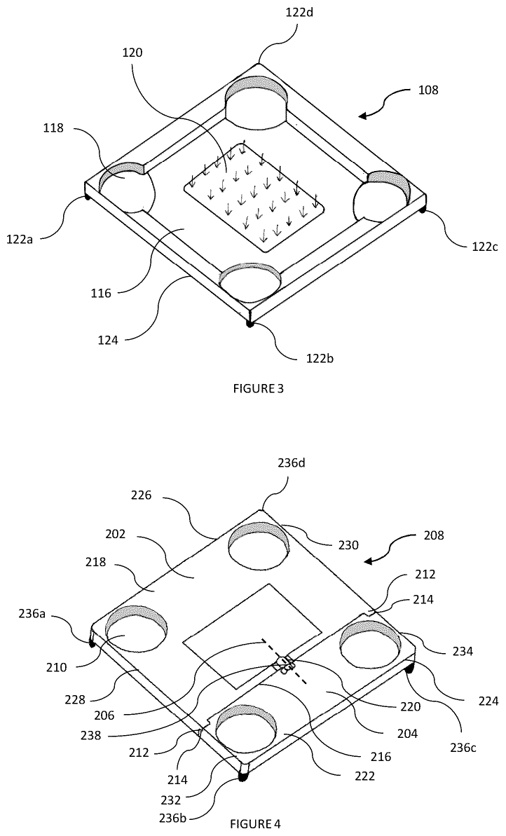

The securing of the surgical cart 104 in a desired position is actuated by a mechanical brake system which is coupled to the base 108. The arrangement and execution of the mechanical brake system is demonstrated in FIGS. 2A and 2B, which illustrate a lower portion of the surgical cart 104 attached to its corresponding base 108 and moveable elements 110. FIG. 2A illustrates the configuration of the lower portion of the surgical cart 104 before the mechanical brake system has been actuated. That is, in FIG. 2A the surgical cart is free to move relative to the floor surface on which it is in contact. FIG. 2B illustrates the configuration of the lower portion of the surgical cart 104 after the mechanical brake system has been actuated. In this second configuration, the cart 104 and the surgical robot 100 as a whole are secured in a desired position.

The mechanical brake system forms part of the surgical robot 100 and comprises a skirt 112. The skirt 112 is located between the main body of the surgical cart 104 and the base 108, such that the main body of the cart is coupled to the base by the skirt. The skirt 112 comprises an outer surface which extends around and downwardly of the main body of the cart. The outer surface of the skirt 112 also extends outwardly from the main body of the cart as its distance from the main body of the cart 104 increases, such that the area encompassed by the lower surface of the skirt 112 is greater than the area encompassed by the bottom surface of the cart 104 from which the skirt extends. In FIG. 2A the base 108 which is coupled to the skirt 112 therefore also has a greater surface area than the main body of the cart 104. This increased surface area acts to improve the stability of the surgical robot 100 when the base 108 is in contact with the floor surface.

The mechanical brake system further comprises a linear lifting column 114. The linear lifting column 114 is rigidly connected to the skirt 112 of the surgical cart 104 and is slidably connected to the main body of the surgical cart 104. The linear lifting column 114 is therefore also connected to the base 108 via the skirt 112. The linear lifting column 114 has a smaller cross-sectional area than the main body of the cart 104. That is, the area of the lifting column 114 in a plane that is parallel to the lower surface of the skirt 112 is smaller than the corresponding area of the main body of the surgical cart 104. This smaller surface area means that, in the configuration illustrated in FIG. 2A, the linear lifting column 114 can be retracted within the main body of the surgical cart 104. In one example, the linear lifting column 114 is hollow, such that the internal area of the surgical cart 104 is minimally reduced by the presence of the column. In an alternative example, the linear lifting column 114 is solid.

In FIG. 2A, movement of the surgical robot 100 relative to the floor surface on which it is in contact is enabled because the moveable elements 110 that are coupled to the surgical cart 104 are exposed. The moveable elements 110 can therefore be used to move the robot.

FIG. 2B illustrates the configuration of the lower portion of the surgical cart 104 when the mechanical brake system is activated. The activation of this system involves lowering the linear lifting column 114 so that it protrudes past the main body of the cart 104. The lowering of the lifting column results in a lowering of the skirt 112 and the base 108 which is coupled to the skirt. The lifting column 114, the skirt 112 and the base 108 are not directly connected to the moveable elements 110. Thus, the arrangement of the lifting column 114, the skirt 112 or the base 108 can be moved independently of the moveable elements 110. That is, the moveable elements 110 can be held stationary and the lifting column 114 can be lowered until the lowermost surface of the base 108 extends past than the bottom of the moveable elements. The lowering of the lifting column 114 therefore results in the moveable elements 110 being lifted off the floor surface, such that the base 108 is instead used to support the load of the surgical robot. In addition to the lowering of the lifting column 114, the mechanical brake system may be configured to actively retract the moveable elements 110 to assist in their separation from the floor surface. This retraction may be performed simultaneously with the lowering of the lifting column 114. The retraction may alternatively be performed in advance or after the lowering of the lifting column 114.

In one example, the mechanical brake system is activated by an operator. In this example the mechanical brake system may be connected to an interface such as a button or a lever that enables the system to be actuated by the operator. In an alternative example, the mechanical brake system may be automatically activated. The interface may be located at the surgeon's console, or alternatively on the surgical cart 104. In one example, the mechanical brake system comprises integrated sensors that are configured to detect when the base 108 has contacted a floor surface. In one example, one or more force sensors are located on the lowermost surface of the base 108 such that they can detect when the base comes into contact with a floor surface. In other examples, alternative types of sensors may be used to detect when the base has contacted a floor surface. Once the base 108 has contacted the surface, the integrated sensors can provide feedback to the controllers of the mechanical brake system to indicate that the act of lowering of the linear lifting column 114 can be terminated. In another example, the mechanical brake system is configured so that the lifting column 114 can only be lowered to a predetermined maximum distance from the body of the surgical cart 104. In a further example, the lowering of the lifting column 114 may be both activated and terminated by an operator. That is, activation and termination of the brake mechanism may be actuated at a surgeon's console, or alternatively via an interface on the surgical cart 104.

During deactivation of the mechanical brake system, the lifting column 114 is raised which results in a raising of the skirt 112 and the base 108 until the moveable elements 110 are exposed. This action may optionally be synchronised with or supplemented by the active deployment of the moveable elements 110.

FIG. 3 illustrates one example of a base 108 that is configured to support a surgical robot. The base 108 can be coupled to the bottom of a surgical cart as illustrated in FIGS. 1, 2A and 2B. The base 108 is a substantially planar, or flat, element. That is, the base 108 has a length and width that are substantially greater than its height. The base 108 can therefore be alternatively described as a plate. The base 108 comprises an upper surface 116 to which a surgical cart can be mounted. The upper surface 116 may be of any suitable shape. In one example, the upper surface is shaped as a quadrilateral. In a more specific example, the upper surface is shaped as a square. As described above, the base 108 is configured for attachment to a skirt 112 of a surgical cart. Thus, in a preferred example the shape of the upper surface of the base 108 is complimentary to that of the lower surface of the skirt 112. In an alternative example, the area of the upper surface 116 is greater than the lower surface of the skirt 112.

The base 108 illustrated in FIG. 3 comprises one or more apertures 118, which are configured such that a moveable element can extend through each aperture. In this example, the base 108 comprises four apertures, which correspond to four moveable elements that are configured to move the surgical cart 104. In alternative examples, it would be appreciated that the surgical cart may comprise more or fewer than four moveable elements. As such, the base 108 may comprise more or fewer than four apertures 118. The one or more apertures 118 allow the base 108 to move independently of the moveable elements so that the elements can either be hidden or exposed by the base 108 during actuation of the mechanical brake system.

The base 108 may be comprised of two distinct layers. The body of the base may be constructed from a metal. In one example, the body of the base is made from steel, such as a non-alloy steel. The metal body provides the base 108 with its structural integrity so that it is able to support a vertical load 120 that is imparted on it by the surgical robot. The base may further comprise an over-moulded polymer layer that surrounds the outer surfaces of the metal body. In one example, the polymer layer is constructed from rubber. The polymer layer is configured to protect the metal body of the base 108 from corrosion and assist with the cleaning of the surgical cart 104.

As mentioned above, the function of the base 108 is to provide structural support for the surgical robot. The base 108 is therefore configured to react to a number of distinct loads that result from the movement of the linear lifting column 114, the weight of the surgical cart 104 and the general movement of the robot 100. When the mechanical brake system is activated such that the base 108 is in contact with a floor surface on which the surgical robot is in contact, the vertical load 120 applied to the upper surface 116 base by the weight of the robot is counteracted by reaction forces located at each of a plurality of resting points 122a-d that are located along the edges of a lower surface 124 of the base 108. The lower surface opposes the upper surface 116 of the base 108. In FIG. 3, where the upper surface (and corresponding lower surface) of the base 108 has the shape of a square, the plurality of resting points 122a-d comprises four resting points are located at each of the four corners of the lower surface 124. In other examples, the base 108 may comprise an alternative number of resting points. The plurality of resting points may not be located at the corners of the lower surface 128.

A disadvantage associated with the arrangement of the base 108 is that its flexibility is very low. In other words, when the base is in contact with an uneven floor surface it may not be able to adapt to the surface in order to maintain the stability of the surgical robot. Consider, for example, that resting points 122a and 122b are located over a first portion of a surface which is more elevated than a second portion of a surface over which resting points 122c and 122d are located. The difference in elevations between the first and second portions may be such that resting points 124c and 124d are unable to contact the second portion of the surface without tilting the base and the corresponding surgical robot. Thus, the elevated first portion of the surface causes a moment about the resting points 122a and 122b, resulting in tilting of the surgical cart, and subsequently the robot arm. In an alternative example, the first, elevated, portion of the surface is located under the centre of the cart and the second, lower, portion of the surface is located under resting points 122a-d. In this example, the uneven surface causes a moment which results in a rotation of the surgical cart about its centre. In both examples, the orientation of the arm is compromised such that any subsequent accidental movement of the cart will be translated into a rotation or destabilisation.

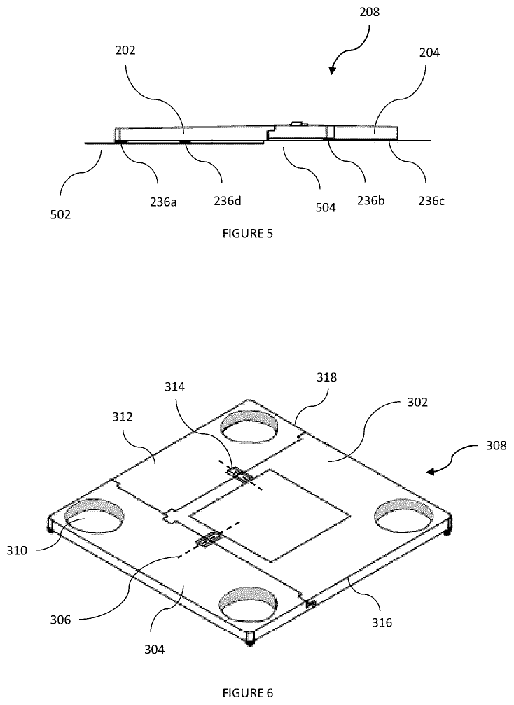

FIG. 4 illustrates a preferred example of a base 208 for supporting a surgical robot, which provides increased stability advantages over the base illustrated in FIG. 3. The material composition of the base 208 is the same as that described above with respect to FIG. 3. As with the example illustrated in FIG. 3, the base is a substantially planar, or flat, element. That is, the base 208 has a length and width that are substantially greater than its height. The base 208 can therefore be alternatively described as a plate.

The base 208 comprises a first planar member 202 and a second planar member 204. The first and second planar members are separated by a line 216 that extends between the members. The first planar member 202 is configured to support the surgical robot. That is, the surgical robot is mounted on the first planar member 202. More specifically, the surgical cart of the robot is mounted on the first planar member 202. The surgical robot is not mounted on the second planar member 204, but this member is pivotally coupled to the first planar member 202 about a first axis 206. The first axis 206 enables the independent rotation of the first planar member 202 and the second planar member 204 about the first axis 206. This independent rotation of the first and second planar members about the first axis 206 acts to stabilise the base on an uneven floor surface. The first axis 206 may be perpendicular to the line 216 extending between the first and second planar members.

The first planar member 202 comprises an upper surface 218, to which the surgical cart is mounted. The second planar member 204 comprises an upper surface 222. As the first planar member 202 is the member on which the cart is mounted, the area of the upper surface 218 of the first planar member may be greater than the area of the upper surface 222 of the second planar member. In one example, when the base 208 is in contact with an even floor surface, the upper surface 222 of the second planar member is aligned with the upper surface 218 of the first planar member. That is, the upper surface 218 of the first planar member is located in the same plane as the upper surface 222 of the second planar member. In this example, the combined upper surfaces 218, 222 of the first and second planar members form a combined upper surface of the base 208.

In one example, at least one distal edge of the first planar member 202 is aligned with at least one distal edge of the second planar member 204. That is, in FIG. 4 a first distal edge 228 of the first planar member is aligned with a first distal edge 232 of the second planar member.

A second distal edge 230 of the first planar member is also aligned with a second distal edge 234 of the second planar member. Thus, the upper surface of the base 208 forms one coherent shape.

Both the upper surface 218 of the first planar member and the upper surface 222 of the second planar member may be shaped as quadrilaterals. The first axis 206 may be parallel to at least one distal edge of the first planar member. In FIG. 4, the first axis 206 is parallel to the first distal edge 228 of the first planar member. The first axis 206 is also parallel to the second distal edge 230 of the first planar member, which opposes the first distal edge. The first axis 206 is also parallel to the first distal edge 232 of the second planar member, and to the second distal edge 234 of the second planar member. The alignment of the first axis 206 so that it is parallel to at least one distal edge of the first or second member assists with the construction of the base 208. It would be appreciated that in alternative examples the first axis 206 may be at an angle to a distal edge of either the first or second planar members. The first axis 206 may further lie on a plane that contains the first planar member 202. That is, the first axis 206 may intersect the first planar member. In a more specific example, the first axis 206 may lie on the upper surface 218 of the first planar member.

The upper surface of the base that is formed from the combination of upper surfaces 218 and 222 may be of any suitable shape. In one example, where upper surfaces 218 and 222 are quadrilaterals, the combined upper surface of the base 208 is also shaped as a quadrilateral. In a more specific example, and as illustrated in FIG. 4, the combined upper surface of the base 208 is shaped as a square. The base 208 is configured for attachment to a skirt 112 of a surgical cart 104. Thus, in a preferred example the upper surface of the base 108 has a complimentary area to that of the lower surface of the skirt 112. In an alternative example, the area of the upper surface 116 is greater than the lower surface of the skirt 112.

The movement of the first planar member 202 and the second planar member 204 may be constrained such that these members are displaceable relative to each other about only the first axis 206. That is, relative motion of the first planar member 202 and the second planar member 204 may only be enabled by rotation about the first axis 206. The base 208 may comprise a mechanical component 220 that is configured to limit the relative motion of the first and second planar members about only the first axis 206. In one example, this mechanical component is a bearing. It would be appreciated that any type of suitable bearing may be selected as the mechanical component 220. The bearing enables the pivotal coupling of the first planar member 202 to the second planar member 204.

The base 208 may further comprise one or more apertures 210, such that a moveable element is able to extend through each of the apertures. In the example illustrated in FIG. 4, the base 108 comprises four apertures, which correspond to four moveable elements that are configured to move the surgical cart 104. In alternative examples, it would be appreciated that the surgical cart may comprise more or fewer than four moveable elements. As such, the base 208 may comprise more or fewer than four apertures 210. The one or more apertures 210 allow the base 208 to move independently of the moveable elements so that the elements can either be hidden or exposed by the base 208 during actuation of the mechanical brake system.

The base may further comprise an arrangement of one or more limiting features 212, 214. That is, the first planar member may comprise at least one first limiting feature 212, and the second planar member may comprise at least one second limiting feature 214. The first limiting feature 212 may correspond to the second limiting feature 214 such that the first and second limiting features are configured to interact. The interaction of the first and second limiting features may be provided to limit the rotation of the first and second planar members about the first axis 206. That is, as the first and second planar members reach a predetermined degree of rotation in a given direction the first and second limiting features will contact each other, prohibiting any further rotation of the members in that direction. In other words, at the predetermined degree of rotation the maximum range of relative rotation of the first and second planar members has been reached and further rotation is prohibited by the limiting features. These features are particularly useful for limiting the degree of rotation of the first and second planar members when the mechanical brake system is not activated, and the base plate is not in contact with a floor surface.

In FIG. 4, the first limiting feature 212 is a tab extending from the body of the first planar member 202. That is, the tab extends past the line 216 that separates the first planar member 202 from the second planar member 204. The second limiting feature 214 in FIG. 4 is an indent in the second planar member that is complementary to the tab in the first planar member 202. That is, where the tab extends past the line 216 that separates the first planar member 202 the indent retracts behind this line. The tab 212 is configured to interact with the distal edge of its corresponding indent 214 when a maximum allowable degree of rotation of the planar members has been met, therefore limiting any further rotation. It would be appreciated that alternative arrangements that serve to limit the relative rotation of the first and second planar members may be implemented in place of the tab and indent arrangement.

The first planar member 202 may comprise a pair of first limiting features 212, with the second planar member 204 comprising a corresponding pair of second limiting features 214. In FIG. 4, the pair of limiting first features 212 and a corresponding pair of second limiting features 214 are located at either end of the line 216 that separates the first planar member from the second planar member. Thus, the arrangement of the first and second pairs of limiting features is symmetrical about the first axis 206. In an alternative example, the first planar member 202 comprises only one first limiting feature 212 and the second planar member 204 comprises only one corresponding second limiting feature 214. In alternative examples the planar members may each comprise more than two respective limiting features.

The line 216 that separates the first planar element 202 from the second planar element 204 may be parallel to at least one distal edge of the base 208. In the example illustrated in FIG. 4 the line 216 is parallel to two distal edges 224, 226 of the base 208. As described above with respect to the orientation of the first axis 206, this configuration assists with the construction of the base. However, it would be appreciated that in alternative examples the line 216 separating the first planar member from the second planar member may not be parallel to a distal edge of the base. For example, the upper surface of the base may not be in the shape of a quadrilateral and may instead be in the shape of a circle. In this arrangement, the line 216 separating the first and second planar members may not be parallel to a distal edge of the base. Where the first axis 206 is perpendicular to the line 216, the first axis 206 may also not be parallel to that distal edge of the base. That is, if the line 216 is orientated at an oblique angle relative to a distal edge of the base, then the first axis 206 would be orientated at an oblique angle relative to the distal edge of the base that is perpendicular to the orientation of the line 206. It would be appreciated that a number of different shapes may be utilised for the upper surface of the base, and that the line separating the first and second planar members may adopt any angle relative to its distal edges.

The arrangement of the base 208 illustrated in FIG. 4 defines two planes that are formed from the combination of three contact points. The first two contact points of each plane are defined by a pair of resting points, one resting point being located on each of the two planar members. For example, resting point 236a of the first planar member and resting point 236b of the second planar member form a first pair of contact points. Correspondingly, resting point 236c on second planar member and resting point 236d on first planar member form a second pair of contact points. The third contact point is defined by the first axis 206. More specifically, the third contact point is a point 238 that intersects the first axis 206 and the line 216 separating the first planar member from the second planar member. Rotation of the first and second planar members results in rotation of the first and second pairs of contact points around the third contact point, varying the orientation of the respective planes that are defined by these points.

Although the surgical robot is only mounted to the first planar member 202, the load that is received by the base is transferred to the second planar member 204 via the mechanical component 220, so that it is shared between both planar members. This arrangement therefore allows the load from the surgical robot to be distributed throughout the base 208.

FIG. 5 illustrates the advantageous effect of the base illustrated in FIG. 4 when it is located on an uneven floor surface. The floor surface in FIG. 5 comprises a first portion of a lower relative elevation 502 and a second portion of a higher relative elevation 504. That is, the first portion 502 of the floor surface has a lower elevation than the second portion 504.

The second planar member 204 is positioned over the second portion 504 of the floor surface, which has a higher elevation than first portion 502, and so will come into contact with the surface first. That is, at least one of resting points 236b and 236c will be the first of the resting points of the base to come into contact with the surface. If only one of resting points 236b and 236c initially contacts the surface, the second planar member 204 may rotate about the first axis 206 to allow the second of resting points to also contact the surface. The first planar member 202 will also rotate about the first axis 206 until at least one of its resting points 236a, 236d contacts the first portion 204 of the surface. For example, the first planar member 202 may rotate in a clockwise direction so that its resting point 236d contacts the first portion 502 of the surface. This rotation may or may not result in resting point 236a also contacting the first portion 502 of the surface. Thus, the pair of contact points 236c, 236d are in contact with the surface. The third contact point 238 will also be in contact with the surface. Thus, three contact points 236c, 236d and 238 form a plane that is able to support the cart load against the surface. At least the resting point 236b of the second planar member is also in contact with the surface. Thus, the number of points of contact between the base 208 and the surface is maximised, which in turn maximises the stability of the surgical robot that is supported by the base. It should be noted that whilst FIG. 4 illustrates resting points 236a-d as extruding outwardly from the base 208, in an alternative example these resting points may simply be the unextruded corners of the base.

FIG. 6 illustrates a further example of a base 308 for supporting a surgical robot. The base 308 corresponds broadly to the base illustrated in FIG. 4. That is, the base 308 comprises a first planar member on which a surgical robot is to be mounted, a second planar member 304 and a first axis 306 about which the first and second planar members are permitted to independently rotate. The base 302 further comprises one or more apertures 310 through which a moveable element is able to extend. In addition to the first and second planar members 302, 304, the base 308 comprises a third planar member 312. The third planar member 312 is pivotally coupled to the first planar member about a second axis 314. Thus, the first planar member 302 and the third planar member 312 are independently rotatable about the second axis 314. By virtue of this arrangement, the third planar member 312 is also independently rotatable of the second planar member 304. The first, second and third planar members may comprise limiting features corresponding to those described above with reference to FIG. 4. The limiting features are configured to limit the rotation of the first, second and third planar members about their respective axes.

In FIG. 6, the second axis 314 is illustrated as being perpendicular to the first axis 306. Where the upper surface of the base 308 is in the shape of a quadrilateral and the first axis 306 is parallel to a first distal edge 316 of the base, the second axis 314 may be parallel to a second distal edge 318 of the base. However, it would be appreciated that in alternative examples the second axis may not be perpendicular to the first axis. In one alternative, the second axis is parallel to the first axis. The second axis may be orientated at any alternative angle relative to the first axis.

Whilst the specification illustrates examples of a base comprising a maximum of three planar members, it would be appreciated that a base could be configured using any additional number of such members. The addition of planar members would act to further stabilise the surgical robot on uneven floor surfaces.

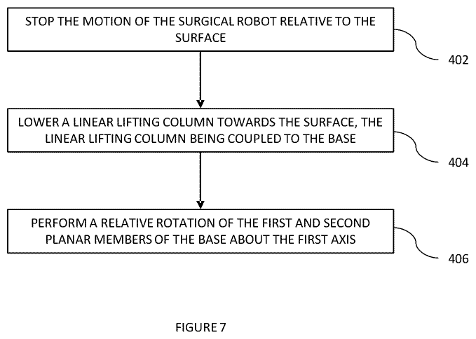

FIG. 7 illustrates a method for stabilising a surgical robot on an uneven floor surface. The surgical robot is as described above with respect to FIG. 1 and incorporates a base as illustrated in either of FIG. 4 or 5. The method is initiated when the surgical robot has been moved to a desired position. The desired position may, for example, be appropriate for commencement of a surgical procedure.

Once the robot has been moved to its desired permission, at step 402 the motion of the surgical robot relative to the floor surface on which it is moving is stopped. The stopping of the surgical robot is instigated by a mechanical brake system. The mechanical brake system is described above with reference to FIGS. 2A and 2B. In one example, the mechanical brake system is activated by an operator. In this example the brake system may be connected to an interface such as a button or a lever than enables the system to be actuated by the operator.

In another example, the brake system may be automatically activated. That is, the robot may be configured to detect when it has reached its desired position and may apply the brake system independently of any input from an operator.

Once the motion of the robot relative to the floor surface has been stopped, then at step 404 the linear lifting column of the mechanical brake system is lowered towards the floor surface. The linear lifting column is coupled to a base as described with reference to FIG. 4 or 5. That is, the base comprises a first planar member on which the surgical robot is mounted and a second planar member pivotably coupled to the first planar member about a first axis. The coupling of the base to the lower surface of the linear lifting column means that the lowering of the lifting column will eventually lead to at least one of the first and second planar members contacting the floor surface.

When at least one member of the base contacts the floor surface, at step 406 a relative rotation of the first and second planar members about the first axis is performed. This relative rotation acts to increase the contact between the base and the floor surface on which it is located, as described above with respect to FIG. 5. The relative rotation of the planar members is dependent on the member that first contacts the floor surface. In one example, the first planar member contacts the floor surface before the second planar member. In this example, the second planar member will rotate relative to the first planar member about the first axis until at least one resting point on the second planar member contacts the floor surface. In an alternative example, the second planar member contacts the floor surface before the first planar member. In this example, the first planar member will rotate about the first axis relative to the second planar member until at least one resting point on the first planar member contacts the floor surface. In a further example, where the floor surface is especially uneven, both the first and second planar members may simultaneously rotate relative to each other about the first axis. As the relative rotation of the first and second planar members acts to increase the number of points of contact between the base and the floor surface, this action results in the stabilisation of the surgical robot on the surface.

Whilst the examples of the invention illustrated in FIGS. 1 to 6 indicate that the surgical robot comprises a cart with moveable elements, it would be appreciated by the skilled person that the bases illustrated in FIGS. 4 and 5 can equally be coupled to a surgical cart that does not comprise any moveable elements. In this configuration, whilst the surgical robot cannot be easily moved, its stability can still be optimised by incorporating a flexible base plate that maximises the contact between the robot and the floor surface on which it is in contact.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A base for supporting a surgical robot, the base comprising:
a first planar member on which the surgical robot is mounted; and
a second planar member pivotably coupled to the first planar member about a first wherein the first planar member and the second planar member are independently rotatable about the first axis so as to stabilise the base on an uneven floor surface.

2. The base as claimed in claim 1, wherein the first axis lies on a plane that contains the first planar member.

3. The base as claimed in claim 1, wherein the surgical robot is mounted on an upper surface of the first planar member.

4. The base as claimed in claim 3, wherein the first axis lies on the upper surface of the first planar member.

5. The base as claimed in claim 1, wherein the movement of the first planar member and the second planar member is constrained such that they are displaceable relative to each other about only the first axis.

6. The base as claimed in claim 1, wherein the first axis is parallel to at least one distal edge of the first planar member.

7. The base as claimed in claim 3, wherein the second planar member comprises an upper surface that is aligned with the upper surface of the first planar member when the base is located on an even surface such that the upper surfaces of the first and second planar members form an upper surface of the base.

8. The base as claimed in claim 7, wherein the upper surface of the base is in the shape of a quadrilateral.

9. The base as claimed in claim 6, wherein the at least one distal edge of the first planar member is aligned with at least one distal edge of the second planar member.

10. The base as claimed in claim 1, wherein the first planar member is pivotably coupled to the second planar member by a bearing.

11. The base as claimed in claim 1, wherein the base comprises one or more apertures, such that a moveable element is able to extend through each aperture, each moveable element being configured to move the surgical robot.

12. The base as claimed in claim 11, wherein the one or more apertures comprises four apertures.

13. The base as claimed in claim 1, wherein the first planar member comprises a first limiting feature configured to interact with a corresponding second limiting feature located on the second planar member, the first and second limiting features being configured to interact to limit the rotation of the first and second planar members about the first axis.

14. The base as claimed in claim 13, wherein the first planar member comprises a pair of first limiting features and the second planar member comprises a corresponding pair of second limiting features.

15. The base as claimed in claim 13, wherein the first limiting feature is a tab extending from the first planar member, and the second limiting feature is an indent in the second planar member that is complementary to the tab.

16. The base as claimed in claim 1, wherein the base is coupled to a mechanical brake system.

17. The base as claimed in claim 16, wherein the mechanical brake system comprises a linear lifting column.

18. The base as claimed in claim 1, further comprising a third planar member pivotally coupled to the first member about a second axis, wherein the first planar member and the third planar member are independently rotatable about the second axis.

19. The base as claimed in claim 18, wherein the second axis is perpendicular to the first axis.

20. A method for stabilising a surgical robot on an uneven floor surface, the method comprising:
stopping the motion of the surgical robot relative to the floor surface;
lowering a linear lifting column towards the floor surface, the linear lifting column being coupled to a base, the base comprising a first planar member on which the surgical robot is mounted and a second planar member pivotably coupled to the first planar member about a first axis, so that the base of the surgical robot contacts the floor surface; and
when the base contacts the floor surface, performing a relative rotation of the first and second planar members about the first axis so as to stabilise the base on the uneven floor surface.
